(19) United States
(12) Patent Application Publication
(10) Pub. No.: US 2004/0029946 A1
(43) Pub. Date: Feb. 12, 2004

(54) Pharmaceutical compositions for topical delivery of cyclooxygenase-2 enzyme inhibitors

(76) Inventors: Virod Kumar Arora, New Delhi (IN); Ajay Kumar Singla, Chandigarh (IN); Mukesh Kumar, Punjab (IN)

Correspondence Address: Jayadeep R Deshmukh, Ranbaxy Pharmaceuticals Inc, Suite 2100, 600 College Road East, Princeton, NJ, 08540, US

(21) Appl. No.: 10/363,326

(22) PCT Filed: Aug. 28, 2001
(86) PCT No.: PCT/IB01/01557

(30) Foreign Application Priority Data
Aug. 29, 2000 (IN) 779/DEL/2000

(51) Int. Cl. A61K 31/415; A61K 9/14; A61K 31/365

(52) U.S. Cl. 514/406; 424/486; 424/488; 514/473

(57) ABSTRACT

The present invention relates to a pharmaceutical composition for topical delivery comprising a pharmaceutically effective amount of drug(s) that acts selectively as a cyclooxygenase-2 enzyme inhibitor.

### FIELD OF THE INVENTION

The present invention relates to a pharmaceutical composition for topical delivery comprising a pharmaceutically effective amount of drug(s) that acts selectively as a cyclooxygenase-2 enzyme inhibitor.

### BACKGROUND OF THE INVENTION

Due to availability of large surface area, easy accessibility, application dynamics and the non-invasive nature of the therapy, topical administration of drugs has long been considered a promising route of drug delivery whether the bioavailability desired is systemic, dermal, regional or localized. This mode of drug delivery provides many advantages over customarily used routes of administration. It bypasses the portal circulation and thereby the hepatic first-pass metabolism, avoids the variable systemic absorption and metabolism and also, potentially reduces gastro-intestinal irritation associated with oral administration. Further, it avoids the risks and patient non-compliance associated with parenteral treatment. Topical route offers continuity of drug administration, permits use of therapeutic agents with short biological half-lives, provides treatment of cutaneous manifestations of diseases usually treated systemically, delivers medication directly into the systemic circulation and foster ease of use and total patient compliance.

Host of patents have been granted pertaining to topical compositions of drugs. By way of example, U.S. Pat. No. 5,093,133 discloses a hydroalcoholic gel of pH 3.5-6.0 consisting essentially about 1-15% substantially pure S-ibuprofen, 0-20% of propylene glycol, about 40-60% alcohol, about 2-5% of a gelling agent selected from the group consisting of hydroxypropyl cellulose and polyacrylic acid polymers and about 0.25-2% of triethanolamine to adjust the pH. The rate of delivery of ibuprofen from such a system is allegedly pH dependent. It is believed that such a topical system wherein such high concentration of alcohol is used, repeated application could cause unfavourable conditions.

U.S. Pat. No. 5,976,566 describes the use of 1,3-dioxane and 1,3-dioxolane derivatives or acetal as skin penetration enhancers for NSAIDs. It discloses a substantially neutral ibuprofen containing alcoholic or aqueous alcoholic composition which comprises a skin penetration enhancing effective amount in the range of from about 4-15% of a C₇ to C₁₄-hydrocarbyl substituted 1,3 dioxolane, 1,3-dioxane or acetal, about 0-18% of glycol, at least about 40% of volatile alcohol, base to provide a pH in the range of from about 6.5 to about 8 and, optionally, gelling agent effective to thicken the composition to avoid or minimize run-off when applied to the skin. The penetration enhancers used therein are unstable at lower pH. The invention is particularly adapted only for NSAIDs in substantially neutral salt form (pH 6-8) which allegedly makes the gel formulation stable.

U.S. Pat. No. 4,602,040 describes non-aqueous clear gel and topical cream composition of meclofenamic acid. Essentially, the patent discloses a clear gel formulation of meclofenamic acid in a cosolvent system of a polyethylene glycol ester, water soluble lanolin oil, an alcohol and a thickening agent and a cream formulation which is homogenized emulsion of polyethylene glycol ester, glyceryl or propylene glycol ester, triglyceride and mineral oil.

An anti-inflammatory analgesic gel composition, as disclosed through U.S. Pat. No. 4,393,076, comprises ketoprofen as the active ingredient, a glycol, lower alcohol, water and/or a mixture of a lower alcohol with water, a gel forming agent and optionally, a solubilizing agent and/or nonionic surface active agents as penetration enhancers.

U.S. Pat. No. 5,807,568 describes enhanced delivery of flurbiprofen through topical compositions comprising 0.5 to 10% of active, about 10-80% of a lower alcohol, about 0-25% of a glycol, about 0-5% of a gelling agent, an amount of a pH adjusting agent sufficient to adjust the pH of the composition to a range of from about 2 to less than 4.5 and water in an amount sufficient to make up the composition.

As mentioned above, several pharmaceutical compositions are described in literature for topical application of nonsteroidal anti-inflammatory drugs (NSAIDs) which are known to be the most commonly prescribed group of drugs worldwide for analgesic, antipyretic and anti-inflammatory effects. Adverse reactions, mostly associated with gastrointestinal disturbances such as acidity, ulceration, hepatic and nephric disorders etc. have been reported with repeated oral NSAID therapy. Hitherto, topical application is one of the preferred alternative routes of administration. Direct application to inflammed joints results in appreciably lower systemic blood levels, reduced gastrolesivity and thereby better tolerance.

Further, NSAIDs are known to act through inhibition of cyclooxygenase and lipoxygenase pathway of arachidonic acid metabolism. The cyclooxygenase (COX) enzyme catalyses the first step in the conversion of arachidonic acid to prostanoids (prostaglandins and thromboxanes). The central mechanism leading to the therapeutic effects of NSAIDs is through the blockade of prostagladin synthesis resulting from inhibition of cyclooxygenase enzyme. The gastrointestinal adverse effects of these drugs are also largely attributable to cyclooxygenase inhibition. Recent research has revealed that this enzyme exists in 2-isoforms, COX-1 and COX-2. It is proposed that inhibition of COX-1 results in their shared adverse effects, whilst COX-2 being the primary isoform available at the sites of inflammation, its inhibition accounts for the therapeutic benefits of NSAIDs.

Fuelled by this hypothesis, much of the recent research has focused upon efficacious methods for development of drug delivery of COX-2 enzyme inhibitors to treat inflammation associated maladies.

### SUMMARY OF THE INVENTION

In light of the foregoing, the principal object of the present invention is to provide a process for the preparation of pharmaceutical compositions for topical delivery of COX-2 enzyme inhibitors.

It is a further object of the present invention to provide a process for the preparation of such compositions which provide enhanced skin penetration and achieve therapeutic levels of the COX-2 enzyme inhibitors in target internal tissues.

Also, it is an object of the present invention to provide a process for the preparation of such compositions, with low dermal irritation and skin sensitization.

Yet another object of the present invention is to provide a process for the preparation of such compositions that have good stability and good cosmetic characteristics.

An additional object of the present invention is to provide a vehicle which is suitable for topical application to the skin and that results in rapid penetration of COX-2 enzyme inhibitor dissolved or suspended therein.

In keeping with these objectives, the present invention relates to a pharmaceutical composition containing as drug a cyclooxygenase-2 enzyme inhibitor for topical application, which effects readier solubility of the active ingredient and which transports the active through the barrier of the stratum corneum, and to the use thereof. As embodied and fully disclosed herein, the present invention describes a process for the preparation of a pharmaceutical composition for topical delivery comprising a pharmaceutically effective amount of drug(s) that acts selectively as a cyclooxygenase-2 enzyme inhibitor, from about 0.3% to about 40% of a gelling agent, from about 2% to about 60% of a solubilizing agent, and optionally, a pH modifying agent and/or other pharmaceutically acceptable adjuvants, said percentages being w/w of the composition.

The present invention also comprehends a pharmaceutical composition incorporating COX-2 inhibitor in the carrier base and optional pharmaceutical adjuvants such as penetration enhancers, humectants and/or moisturizers, preservatives, opacifiers, fragrances, color additives, counter-irritants, and the like.

The pharmaceutical compositions of the invention are intended for topical, non-invasive application to the skin, particularly to the region where the COX-2 enzyme inhibitor is intended to exert its pharmacological activity, usually to a region of inflammation, injury or pain, to the muscles or joints, or other forms of cutaneous disorders or distruptions characterized by skin inflammation and/or hyperproliferative activity in the epidermis of skin.

According to the present invention the pharmaceutical compositions is such that it provides release of at least one therapeutic agent or drug. The drug may be pharmacologically active itself or may be converted into the active form by biotransformation in the body. The combination of drugs that are typically administered together may be included as the drug component. However, in embodiments wherein such a combination is used at least one of such drug acts selectively as a cyclooxygenase-2 enzyme inhibitor.

Illustrative examples of the COX-2 enzyme inhibitors that are advantageously administered by the pharmaceutical compositions of this invention include specific inhibitors such as celecoxib, valdecoxib, rofecoxib, varecoxib, parecoxib, and the like or preferential inhibitors such as meloxicam, nimesulide, etodolac, and the like.

In a particular preferred embodiment of the present invention the composition contains celecoxib or rofecoxib as the drug.

The drug itself or its pharmacologically active salt or ester can be used in the present invention. The amount of drug suitable for the present invention is that which is typically administered for a given period of time. This includes a pharmaceutically effective amount of the drug which is an amount high enough to significantly positively modify the condition to be treated, but low enough to avoid serious side effects (at a reasonable benefit/risk ratio), within the scope of sound medical judgement. The precise amount of drug will vary with the specific drug, the ability of the composition to penetrate the drug through the skin, the amount of the composition to be applied, the particular condition being treated, the severity of the condition, the duration of the treatment, the nature of concurrent therapy, the age and physical condition of the patient being treated, and the like factors. Accordingly, the drug dissolved or dispersed therein, may be present in amount ranging from a pharmaceutically effective amount upto 25% by weight of the total weight of the composition.

According to the present invention, the composition contains an agent which provides the desired integral gel structure to the composition. The choice of gelling agents to be used are considered to be within the purview of one skilled in the art, provided they are compatible with the drug, solubilizing agents and other adjuvants.

The gelling agents preferred for the present invention include inorganic and organic macromolecules capable of forming gel structure. They may be of the hydrophilic or the hydrophobic type or pH dependent or pH independent in nature. Examples of gelling agents suitable for this invention include the agents well known in the pharmaceutical art for their gelling properties and may be selected from the group comprising cellulose ethers such as hydroxypropyl cellulose, hydroxyethyl cellulose, hydroxyethyl methylcellulose, methylcellulose, hydroxypropyl ethylcellulose, hydroxypropyl methylcellulose, carboxymethyl cellulose, sodium carboxymethyl cellulose, hydroxycellulose, and the like; vinyl alcohols such as Polyviol or Moviol, and the like; vinyl pyrrolidones such as Kollidon or Plasdone, and the like; natural gums such as karaya gum, locust bean gum, guar gum, gelan gum, xanthan gum, gum arabic, tragacanth, carrageenan, pectin, agar, alginic acid, sodium alginate, and the like; acrylic polymers such as methacrylates such as available as Eudragit and polyacrylates such as available under the brandname Carbopol; polyoxyethylene-polyoxypropylene copolymers (Poloxamer) such as available as Lutrol, and the like.

In a particular preferred embodiment of the present invention, the composition contains polyacrylate or poloxamer as the gelling agent.

The requisite amount of gelling agent used in this invention is an amount needed to obtain a gel formulation of desirable consistency that allows for easy application to the skin. A low concentration of gelling agent makes the formulation loose or fluid which runs on application, while higher concentration results in stiff formulation that are not easily spreadable. The gelling agents may be present from about 0.3% to about 40% or preferably from about 0.5% to about 30% by weight of the total weight of the composition.

According to the present invention, the pharmaceutical composition contains solubilizing agents which aids in the solubility and better penetration of the drug through skin. The solubilizing agents may be volatile, or non-volatile in nature or a combination thereof.

The compositions of the invention may contain a volatile solubilizing agent that includes especially lower alkanols having preferably 2 or 4 carbon atoms such as ethanol, denatured ethanol (commercially available as SDA-40), propanol, isopropanol, butanol and mixtures thereof. Other pharmaceutically acceptable alcohols may also be used in this invention.

According to the present invention, the compositions may comprise non-volatile solubilizing agent. Examples of non-volatile solubilizing agents that may be used in the present invention include glycols and derivatives thereof such as butylene glycol, propylene glycol, polypropylene glycol, polyethylene gycol, hexylene glycol, polyethylene glycol dodecyl ether, diethylene glycol monoethyl ether (available commercially as Transcutol), polyethylene glycol-8 glyceryl caprylate (commercially available as Labrasol), propylene glycol monocaprylate (commercially available as Capryol 90), and the like; polysorbates such as available as Tween 20, Tween 40, Tween 60, Tween 80, and the like; Sorbitan esters such as sorbitan monolaurate (Span 20), sorbitan monopalmitate (Span 40), sorbitan monostearate (Span 60), sorbitan trioleate (Span 85), and the like; polyoxyl oil derivatives such as polyoxyl 60 hydrogenated castor oil (available as Cremophor RH40), polyoxyl castor oil, polyoxyl 35 castor oil, polyoxyl 40 hydrogenated castor oil, and the like. Other pharmaceutically acceptable solubilizing agents such as dimethyl sulfoxide, dimethyl formamide, benzyl alcohol, and the like may also be used. These solubilizing agents may be used alone or in a mixture of at least two or more.

The total amount of the solubilizing agent used, depends on the factors such as amount of COX-2 inhibitor, type of COX-2 inhibitor, amount and nature of gelling agent, and the like. However, the composition of the invention may contain solubilizing agents in an amount from about 2% to about 60%, preferably from about 5% to about 50% and more preferably from about 10% to about 40% by weight of the total weight of the composition.

In preferred embodiment of the present invention the pharmaceutical composition contains combination of ethanol, polyethylene glycol-8 glyceryl caprylate, polyethylene glycol and propylene glycol as the solubilizing agents.

These compositions containing alcohol are of great utility in solubilizing active ingredients which are poorly soluble in glycol but highly soluble in alcohol. Moreover, the alcohol contained in the composition exerts a bactericidal and bacteriostatic effects on skin areas to which the compositions are applied, and provides a cooling counter-balance to the glycol solubilizing agents which may sometimes create a warming sensation when applied to the skin. The solubilizing agents disclosed herewith provide unique advantages. Such a system provide stable non-irritating composition of a wide variety of drugs and aids in penetration of COX-2 enzyme inhibitors with even high molecular weights through the skin.

The interplay of alcohol and glycols as a solubilizing agent improves solubility of polar drugs and those that are primarily sparingly soluble in water. In addition, such a combination promotes improved resorbability of the COX-2 enzyme inhibitor. Further, such a combination improves spreadability and aestethics of the pharmaceutical composition. It minimizes any congealing or balling up or drying of the composition when it is rubbed on the skin. Furthermore, polyethylene glycol-8 glyceryl caprylate being a surfactant acts as a permeation enhancer and hence improves penetration of the COX-2 enzyme inhibitor. Also, such a combination gives better consistency, as ethanol or polyethylene glycol-8 glyceryl caprylate or polyethylene glycol alone results in a composition with high fluidity, whilst propylene glycol alone results in a tacky composition, which does not spread uniformly.

According to the present invention, the compositions may also comprise a pH modifying agent. The present invention is directed to a pharmaceutical composition exhibiting an optimal flux or diffusion for the topical delivery of COX-2 enzyme inhibitors. It is well known to the one skilled in art that composition at optimal pH maximizes the flux i.e. the rate of delivery of the drug through skin. Further, most gelling agents usable in accordance with the present invention are highly acidic which drop the pH below the desirable range. Furthermore, certain gelling agents in accordance with the present invention forms integral gel structure only at near neutral pH. Carboxyvinyl polymers is one such example. These are hydrophillic polymers that are prepared by polymerizing monomers principally consisting of acrylic acid. Due to the presence of free carboxylic acid residues, an aqueous solution of this polymer is acidic in nature. Neutralization of this solution cross-links and gelatinizes the polymer to form a viscous integral structure of desired viscosity.

Accordingly, any well known and pharmacologically safe inorganic or organic basic compounds can be used for modifying the pH. Examples of inorganic basic salts that may be used in the present invention include ammonium hydroxide, alkali metal salts, alkaline earth metal salts such as magnesium oxide, magnesium hydroxide, calcium hydroxide, sodium hydroxide, potassium hydroxide, lithium hydroxide, aluminium hydroxide, potassium carbonate, sodium bicarbonate, and the like. The examples of organic basic salts that may be used in the present invention include alkanolamines such as methanolamine, ethanolamine, propanolamine, butanolamine, dimethanolamine diethanolamine, dipropanolamine, dibutanolamine, diisopropanolamine, trimethanolamine triethanolamine, tripropanolamine, diisopropanolamine, tributanolamine, aminomethylpropanol, N-methyl glucamine, tetrahydroxypropyl ethylene diamine, and the like; alkylamines such as methylamine, ethylamine, propylamine, butylamine, diethylamine, dipropylamine, isopropylamine, and the like.

In preferred embodiment of the present invention the pharmaceutical composition contains triethanolamine as the pH modifying agent.

For any particular composition, the drug and likewise the other ingredients may be selected to achieve the desired release profile and the extent of penetration. The optimum pH may then be determined and will depend on factors such as nature of COX-2 enzyme inhibitor, gelling agent, degree of flux required, and the like. However, the pH of the pharmaceutical composition according to the present invention may be between 3.0 and 8.0, and preferably between 4.0 and 7.0.

Optionally, there may also be incorporated into the pharmaceutical composition of the present invention other conventional pharmaceutically acceptable adjuvants known in the art of formulation development such as penetration enhancers, humectants and/or moisturizers, preservatives, opacifiers, fragances, color additives, counter-irritants and the like. The adjuvants selected should be such that there is no interaction which would substantially reduce the pharmaceutical efficacy of the composition of the present invention. Pharmaceutical adjuvants used must be of high purity and low toxicity to render them suitable for administration.

The composition of the invention may further comprise penetration enhancers for improved transepidermal or percutaneous delivery of drug. The penetration enhancers suitable for the present invention include terpenes, terpene alcohols, essential oils, surfactants, and the like. Some such examples include d-limonene, terpinen-4-ol, menthone, 1,8-cineole, 1-pinene, α-terpineol, carveol, carvone, pulegone, eucalyptol, peppermint oil, sorbitan esters, polysorbates, sodium lauryl sulphate, and the like.

The pharmaceutical compositions in accordance with the present invention may also contain one or more humectants and/or moisturizers. These may include polyhydroxy alcohols such as sorbitol, glycerin, hexanetriol, butanediol, mannitol, glucose, ethylene glycol, propylene glycol, and the like.

Preservatives such as methylparaben, propylparaben, phenoxyethanol, benzyl alcohol, bromopol, chlorocresol, thiomersal, benzalkonium chloride, and the like may be added to the compositions to inhibit microbial activity.

Opacifiers, such as behenic acid, glycol distearate, lard glycerides, polyethylene glycol esters, and the like; fragrances such as amyl salicylate, p-anisaldehyde, anisylalcohol, peppermint oil, wintergreen oil, and the like; colour additives such as quinoline yellow, and the like; counter-irritants such as methyl salicylate, menthol and the like; and other pharmaceutical adjuvants may be added to the compositions of the invention.

Preferably, the composition of the present invention may have a viscosity of within the range of about 50,000 to 3.5 million centipoises (cps), preferably between about 300,000 to 2.5 million cps, and even more preferably between about 800,000 to 2.0 million cps, when measured using a Brookfield type RVT series viscometer with helipath stand at ambient temperature (20° C.) and with a 0.5 inch helipath and T-spindle (size “E”) rotating at 2.5 RPM in a sample size ranging from 90-100 grams.

The compositions hereof have good stability. They do not show any substantial changes in viscosity at high temperatures or crystallization at low temperatures. Moreover, they adhere well to the skin and spread readily. Further, they do not impart a sticky feeling and dry easily.

The in vitro release profiles were characterized using modified Franz diffusion cells consisting of two compartments, a donor and a receptor, separated by a cellulose acetate nitrate (0.45μ) membrane on which a thin layer of test product was uniformly spread, whilst isopropyl alcohol and water mixture was used as a medium to maintain the sink conditions in the receptor compartment. The cellulose acetate nitrate membrane hinders the penetrant as it diffuses through its channels and the transport process correlates at best with molecular permeation across porous capillary endothelium. However, the transport mechanism is diffusion or passage through macroscopic ducts filled with solvent. All studies were conducted at 32° C.

### DETAILED DESCRIPTION OF THE INVENTION

The following examples further illustrate this invention, and are not to be construed as limiting the same but read in conjunction with the description above, provide further understanding of the present invention and an outline of the process for preparing the compositions of the invention.

### EXAMPLE 1

This example illustrates the preparation of pharmaceutical composition using carboxyvinyl polymer as the gelling agent in conjuction with the solubilizing agent comprising glycols, alcohol and surfactant. The active ingredient is celecoxib. The pharmaceutical composition is given below in Table 1.

TABLE 1

| Ingredients | Quantity (% w/w) |
|---|---|
| Celecoxib | 3.0 |
| Carboxypolymethylene (Carbopol 940) | 1.0 |
| Polyethylene glycol (PEG- 400) | 15.0 |
| Propylene Glycol | 5.0 |
| Polyethylene glycol-8 glyceryl | 10.0 |
| Caprylate (Labrasol) | |
| Ethanol | 10.0 |
| Triethanolamine | 1.0 |
| Phenoxyethanol | 1.0 |
| Fragrance (Oil of lemon lime) | 0.4 |
| Purified water | to |
| | 100 |

Polyethylene glycol, propylene glycol, polyethylene glycol-8 glyceryl caprylate, phenoxyethanol and a portion of water (about 200 ml) were stirred well to form a dispersion. Celecoxib, was then added slowly under continuous stirring. The stirring was continued till a uniform dispersion was formed. Carboxyvinyl polymer was further dispersed in the resultant dispersion following which ethanol and fragrance was also added. Triethanolamine dissolved in a portion of water (about 50 ml) was then added which initiated viscous structure formation. The weight was made upto 500 g with purified water and the resultant mixture was thoroughly mixed until had wholly been made homogenous to obtain an anti-inflammatory analgesic topical composition. The resultant composition had a pH of 5.83 and a viscosity of 1,62,000 cps.

The composition was studied for in vitro release profile using modified Franz diffusion cells. The samples of the receptor media (IPA:Water::55:45) were analyzed for celecoxib content at regular intervals, spectrophotometrically. The results are shown in Table 2.

TABLE 2

| Time (Min) | Flux (μg/cm²) |
|---|---|
| 15 | 1.268 |
| 30 | 3.325 |
| 60 | 5.513 |
| 120 | 7.714 |
| 180 | 8.536 |
| 240 | 8.837 |

### EXAMPLE 2

This example illustrates the preparation of pharmaceutical composition using carboxyvinyl polymer as the gelling agent in combination with glycols, alcohols and surfactant as solubilizing agents. The active ingredient is Rofecoxib. The pharmaceutical composition is given below in Table 3.

TABLE 3

| Ingredients | Quantity (% w/w) |
|---|---|
| Rofecoxib | 1.0 |
| Carboxypolymethylene (Carbopol 940) | 1.0 |
| Polyethylene glycol (PEG- 400) | 15.0 |
| Propylene Glycol | 5.0 |
| Polyethylene glycol-8 glyceryl | 10.0 |
| Caprylate (Labrasol) | |
| Ethanol | 10.0 |
| Triethanolamine | 0.5 |
| Phenoxyethanol | 1.0 |
| Fragrance (Oil of lemon lime) | 0.4 |
| Purified water | to |
| | 100 |

Polyethylene glycol, propylene glycol, polyethylene glycol-8 glyceryl caprylate and phenoxyethanol were stirred well to form a dispersion. Rofecoxib was then added slowly under continuous stirring. The stirring was continued till a uniform dispersion was formed. Carboxyvinyl polymer was further dispered in the resultant dispersion following which a portion of water was added. Ethanol, fragrance and a solution of triethanolamine was then dispersed. The weight was made upto 500 g with purified water and the resultant mixture was thoroughly agitated until a homogenous composition was obtained. The resultant composition had a pH of 5.87 and a viscosity of 1,50,000 cps.

The composition was studied for in vitro release profile using modified Franz diffusion cell and the samples of the receptor media (IPA:Water::70:30) were analyzed for rofecoxib content at prescheduled timings, spectrophotometrically. The results are tabulated in Table 4.

TABLE 4

| Time (Min) | Flux (μg/cm²) |
|---|---|
| 15 | 3.495 |
| 30 | 5.962 |
| 60 | 10.303 |
| 120 | 13.665 |
| 180 | 14.970 |
| 240 | 17.015 |

### EXAMPLE 3

This example illustrates the preparation of pharmaceutical composition using carboxyvinyl polymer as the gelling agent in combination with a solubilizing agents containing only glycols and alcohol. The pharmaceutical composition is given below in Table 5.

TABLE 5

| Ingredients | Quantity (% w/w) |
|---|---|
| Rofecoxib | 1.0 |
| Carboxypolymethylene (Carbopol 940) | 1.0 |
| Polyethylene glycol (PEG- 400) | 15.0 |
| Propylene Glycol | 5.0 |
| Ethanol | 10.0 |
| Triethanolamine | 0.5 |
| Phenoxyethanol | 1.0 |
| Fragrance (Oil of lemon lime) | 0.4 |
| Purified water | to |
| | 100 |

The pharmaceutical composition was prepared as described in Example 2. The composition with a pH of 5.82 and a viscosity of 1,40,000 cps was obtained.

The composition was studied for in vitro release profile as described in Example 2. The results are tabulated in Table 6.

TABLE 6

| Time (Min) | Flux (μg/cm²) |
|---|---|
| 15 | 2.320 |
| 30 | 4.327 |
| 60 | 6.561 |
| 120 | 11.096 |
| 180 | 15.034 |
| 240 | 16.283 |

### EXAMPLE 4

This example illustrates the use of polyoxyethylene-polyoxypropylene copolymer as the gelling agent. The pharmaceutical composition is given in Table 7.

TABLE 7

| Ingredients | Quantity (% w/w) |
|---|---|
| Celecoxib | 3.0 |
| Polyoxyethylene-polyoxypropylene copolymer | 25.0 |
| (Poloxamer 407, Lutrol) | |
| Polyethylene glycol (PEG- 400) | 15.0 |
| Propylene Glycol | 5.0 |
| Polyethylene glycol-8 glyceryl | 10.0 |
| Caprylate (Labrasol) | |
| Ethanol | 10.0 |
| Phenoxyethanol | 1.0 |
| Fragrance (Oil of lemon lime) | 0.4 |
| Purified water | to |
| | 100 |

Polyethylene glycol, propylene glycol, polyethylene glycol-8 glyceryl caprylate, ethanol and phenoxyethanol were stirred to form a clear dispersion. Celecoxib was then added slowly under continuous stirring. The stirring was continued till a clear solution was obtained. Polyoxyethylene-polyoxypropylene copolymer (Lutrol) was heated to 60-70° C. This was cooled to 50° C. and the drug solution prepared above, was added to lutrol base under continuous stirring. Fragrance was then dispersed and purified water was added. The resultant mixture was stirred well till a homogenous clear composition of 500 g was obtained.

The resultant composition had a pH of 5.97 and a viscosity of 1,000,000 cps.

The composition was studied for in vitro release characteristics as described in Example 1. The results are shown in Table 8.

TABLE 8

| Time (Min) | Flux (μg/cm²) |
|---|---|
| 15 | 1.393 |
| 30 | 5.297 |
| 60 | 10.785 |
| 120 | 30.074 |
| 180 | 60.142 |
| 240 | 72.838 |

While this invention has been described with an emphasis upon preferred embodiments, it will be obvious to those of ordinary skill in the art that variations in the preferred methods of the present invention may be used and that it is intended that the invention may be practiced otherwise than as specifically described herein. Accordingly this invention includes all modifications encompassed within the spirit and scope of the invention as defined by the following claims.

What is claimed is:

1. A pharmaceutical composition for topical delivery comprising a pharmaceutically effective amount of drug(s) that acts selectively as a cyclooxygenase-2 enzyme inhibitor, from about 0.3% to about 40% of a gelling agent, from about 2% to about 60% of a solubilizing agent, and optionally a pH modifying agent and/or other pharmaceutically acceptable adjuvants, said percentages being w/w of the composition.

2. The composition of claim 1 wherein the drug is selected from the group consisting of celecoxib, rofecoxib, varecoxib, parecoxib, valdecoxib, etodolac, nimesulide and meloxicam.

3. The composition of claim 2 wherein the drug is celecoxib.

4. The composition of claim 2 wherein the drug is rofecoxib.

5. The composition of claim 1 wherein the drug is present in an amount upto 25% by weight of said composition.

6. The composition of claim 1 wherein the gelling agent comprises a cellulose ether, vinyl alcohol, vinyl pyrrolidone, natural gum, acrylic polymer, polyoxyethylene-polyoxypropylene copolymer and mixtures thereof.

7. The composition of claim 6 wherein the cellulose ether is selected from the group consisting of hydroxypropyl cellulose, hydroxyethyl cellulose, hydroxyethyl methylcellulose, methyl cellulose, hydroxypropyl ethylcellulose, hydroxypropyl methylcellulose, carboxymethyl cellulose, sodium carboxymethyl cellulose, hydroxycellulose and mixtures thereof.

8. The composition of claim 6 wherein vinyl alcohol is polyvinyl alcohol.

9. The composition of claim 6 wherein vinyl pyrrolidone is polyvinylpyrrolidones.

10. The composition of claim 6 wherein natural gum is selected from the group consisting of karaya gum, locust bean gum, guar gum, gelan gum, xanthan gum, gum arabic, tragacanth carrageenan, pectin, agar, alginic acid, sodium alginate and mixtures thereof.

11. The composition of claim 6 wherein the acrylic polymer is selected from the group consisting of methacrylates, polyacrylates copolymers and mixtures thereof.

12. The composition of claim 6 wherein polyoxyethylene-polyoxypropylene copolymer is poloxamer.

13. The composition of claim 1 wherein the gelling agent comprises about 0.5% to about 30% by weight of said composition.

14. The composition of claim 1 wherein the solubilizing agent comprises a volatile agent, non-volatile agent and mixtures thereof.

15. The composition of claim 14 wherein the volatile solubilizing agent is selected from the group consisting of ethanol, denatured ethanol, propanol, isopropanol, butanol and mixtures thereof.

16. The composition of claim 14 wherein the non-volatile solubilizing agent comprises a glycol and derivatives thereof, polysorbate, sorbitan ester, polyoxyl oil derivatives and mixtures thereof.

17. The composition of claim 16 wherein the glycol is selected from the group consisting of butylene glycol, propylene glycol, polypropylene glycol, polyethylene glycol, hexylene glycol, polyethylene glycol dodecyl ether, diethylene glycol monoethyl ether, polyethylene glycol-8 glyceryl caprylate, propylene glycol monocaprylate and mixtures thereof.

18. The composition of claim 16 wherein the polysorbate is selected from the group consisting of polyoxyethylene sorbitan monolaurate, polyoxyethylene sorbitan monopalmitate, polyoxyethylene sorbitan monostearate, polyoxyethylene sorbitan monooleate, polyoxyethylene sorbitan trioleate and mixtures thereof.

19. The composition of claim 16 wherein the sorbitan ester is selected from the group consisting of sorbitan monolaurate, sorbitan monopalmitate, sorbitan, monostearate, sorbitan monooleate, sorbitan sesquioleate, sorbitan trioleate and mixtures thereof.

20. The composition of claim 16 wherein the polyoxyl oil derivative is selected from the group consisting of polyoxyl castor oil, polyoxyl 35 castor oil, polyoxyl 40 hydrogenated castor oil, polyoxyl 60 hydrogenated castor oil and mixtures thereof.

21. The composition of claim 1 wherein the solubilizing agent comprises about 10% to about 40% by weight of said composition.

22. The composition of claim 1 wherein the pH modifying agent is an inorganic basic salt or an organic basic salt.

23. The composition of claim 22 wherein the inorganic basic salt is selected from the group consisting of ammonium hydroxide, magnesium oxide, magnesium hydroxide, calcium hydroxide, sodium hydroxide, potassium hydroxide, lithium hydroxide, aluminium hydroxide, potassium carbonate, sodium bicarbonate and mixtures thereof.

24. The composition of claim 22 wherein the organic basic salt is an alkanolamine or alkylamine.

25. The composition of claim 24, wherein the alkanolamine is selected from the group consisting of methanolamine, ethanolamine, propanol-amine, butanolamine, dimethanolamine, dibutanolamine, trimethanolamine, triethanolamine, tripropanolamine, diisopropanolamine, tributanolamine, aminomethyl propanol, N-methyl glucamine, tetrahydroxy propylethylene diamine and mixtures thereof.

26. The composition of claim 24 wherein the alkylamine is selected from the group consisting of methylamine, ethylamine, propylamine, butylamine, diethylamine, dipropylamine, isopropylamine and mixtures thereof.

27. The composition of claim 1 wherein the composition may have a pH between 3.0 and 8.0

28. The composition of claim 1 wherein the pharmaceutically acceptable adjuvants comprises penetration enhancers, humectants and/or moisturizers and preservatives.

29. The composition of claim 28 wherein the penetration enhancer is a terpene, terpene alcohol, essential oils and surfactants.

30. The composition of claim 29 wherein the penetration enhancer may be selected from the group consisting of d-limonene, terpinen-4-ol, menthone, 1,8-cineole, 1-pinene, α-terpineol, carveol, carvone, pulegone, eucalyptol, peppermint oil, sorbitan esters, polysorbates, sodium lauryl sulphate and mixtures thereof.

31. The composition of claim 28 wherein the humectant and/or moisturizer may be selected from the group consisting of sorbitol, glycerin, hexanetriol, butanediol, mannitol, glucose, ethylene glycol, propylene glycol and mixtures thereof.

32. The composition of claim 28 wherein the preservative may be selected from the group consisting of methylparaben, propylparaben, phenoxyethanol, benzyl alcohol, bromopol, chlorocresol, thiomersal, benzalkonium chloride and mixtures thereof.

33. The composition of claim 28 wherein the composition further comprises opacifiers, fragrances, colour additives, counter-irritants or mixtures thereof.

34. The composition of claim 1 wherein the composition may have a viscosity between 50,000 and 3.5 million centipoises.

35. The composition of claim 1 wherein the composition is a gel, a spray, an aerosol, a lotion, a cream or an ointment.
